(19) United States
(12) Patent Application Publication
(10) Pub. No.: US 2023/0333072 A1
(43) Pub. Date: Oct. 19, 2023

(54) REVERSE-PHASE HIGH PRESSURE LIQUID CHROMATOGRAPHY METHODS FOR MEASURING AMINO ACIDS, AMMONIUM, AND GLUTATHIONE CONCENTRATIONS IN BIOLOGICAL SAMPLES

(71) Applicant: CHILDREN'S NATIONAL MEDICAL CENTER, Washington, DC (US)

(72) Inventors: Gary CUNNINGHAM, Washington, DC (US); Marshall SUMMAR, Washington, DC (US)

(73) Assignee: CHILDREN'S NATIONAL MEDICAL CENTER, Washington, DC (US)

(21) Appl. No.: 18/301,056

(22) Filed: Apr. 14, 2023

Related U.S. Application Data
(60) Provisional application No. 63/481,200, filed on Jan. 24, 2023.
(60) Provisional application No. 63/481,192, filed on Jan. 24, 2023.
(60) Provisional application No. 63/363,051, filed on Apr. 15, 2022.

(51) Int. Cl. G01N 30/74; G01N 30/88; G01N 30/14; G01N 30/16; G01N 33/68; B01D 15/32

(52) U.S. Cl. CPC G01N 30/88; B01D 15/325; G01N 30/14; G01N 30/16; G01N 30/74; G01N 33/6809; G01N 2030/027

(57) ABSTRACT

A fast and accurate reverse-phase high pressure liquid chromatography (“RP-HPLC”) method for detecting amino acids in small volumes (e.g. less than 50 µL) of a biological sample, such as plasma. An assay for the simultaneous determination of ammonium and primary amino acids using RP-HPLC in samples such as plasma. A method for calculating intercellular volumes from a cell lysate to which a known volume and concentration of a non-naturally occurring amino acid is added.

### CROSS REFERENCE TO RELATED APPLICATIONS

This application claims priority to U.S. Provisional 63/363,051, filed Apr. 15, 2022, to U.S. Provisional 63/481,192, filed Jan. 24, 2023, and to U.S. Provisional 63/481,200, filed Jan. 24, 2023, each of which is incorporated by reference in its entirety.

### BACKGROUND OF THE INVENTION

### Field of the Invention

The invention pertains to the fields of analytic chemistry and biochemistry. Specifically to sensitive and fast methods of reverse-phase high pressure liquid chromatography (“RP-HPLC”).

### BRIEF SUMMARY OF THE INVENTION

One aspect of the invention is a fast and accurate reverse-phase high pressure liquid chromatography (“RP-HPLC”) method for detecting amino acids in small volumes (e.g. less than 50 µL) of a biological sample, such as plasma. This method comprises removing proteins from a sample, for example, using a spin filter and processing the sample using RP-HPLC with O-phthalaldehyde (OPA) as the pre-column derivatization reagent, and ultraviolet (“UV”) detection of amino acids in the processed sample. Compared to conventional RP-HPLC, it provides a convenient, fast, and accurate way to measure amino acids in small volumed samples using widely available equipment and materials. Detecting amino acid concentrations in plasma and other samples is often important for diagnosing or monitoring patients with diseases or conditions involving nutrition or metabolism. This includes patients having inborn errors in metabolism or genetic diseases that alter amino acid levels in the body.

Another aspect of the invention involves a fast, convenient and accurate assay for the simultaneous determination of ammonium and primary amino acids using RP-HPLC in samples such as plasma. This method uses O-Phthalaldehyde (OPA) as a pre-column derivatization reagent and employs UV to detect amino acids and ammonium. The inventors have validated this assay for linearity, accuracy, and precision over a working range from 0-2000 uM. Detecting ammonium is often important for diagnosing or monitoring medical conditions such as those involving the kidney and liver. Thus, biological samples used in the disclosed method may be obtain from subjects having diseases or conditions with elevated ammonium or for diagnosing or monitoring conditions, such as drug or other cancer therapies which cause tissue destruction and release ammonium, as well as patients with inborn errors in metabolism associated with elevated ammonium.

Another aspect of the invention is an assay that uses RP-HPLC to calculate intercellular volumes from a cell lysate to which a known volume and concentration of a non-naturally occurring amino acid is added. This method is application to determining the volumes of red blood cells, leukocytes and other types of cells such as those derived from somatic tissues. It avoids a need to rely on normalizing to protein concentrations in a sample. As shown herein, it can be applied to detection of glutathione. Detection glutathione concentration can be used to diagnose or monitor diseases or conditions associated with glutathione deficiency such as cancer, diseases of aging including Parkinson’s disease and Alzheimer’s disease, cystic fibrosis, and cardiovascular, inflammatory, immune, metabolic, and neurodegenerative diseases. It may also be applied to detect or monitor toxicological phenomena such as those involving binding of glutathione to methyl mercury, other heavy metals, oxidative chemicals, pesticides and herbicides and other environmental pollutants.

Specific embodiments of the disclosure include, but are not limited to, the following.

A method for detecting amino acids in a sample volume of 50 µL or less comprising: (a) removing proteins from a sample, (b) derivatizing the sample obtained from (a) by contacting it with O-Phthalaldehyde (OPA), (c) injecting the derivatized sample from (b) into a RP-HPLC column to produce fractions, and (d) detecting RP-HPLC resolved fractions or components as they elute from the RP-HPLC column by ultraviolet illumination. Amino acids include the proteogenic amino acids and non-proteogenic amino acids. Proteogenic amino acids include valine, isoleucine, leucine, methionine, alanine, proline, glycine, phenylalanine, tyrosine, tryptophan, histidine, asparagine, glutamine, serine, threonine, lysine, arginine, histidine, pyrolysine, aspartic acid, glutamic acid, selenocysteine, and cysteine.

This method may be performed using samples having no volumes more than <10, 15, 20, 25, 30, 35, 40, 45, 50, 60, 70, 80, 90, 100, or >100 µL, such as biological samples having volumes of 25 µL or less.

In some embodiments of this method the samples comprise a biological sample, such as blood, plasma, or serum. In other embodiments, the samples may be derived from liquid or solid tissues in vivo, ex vivo, or in vitro, such as from somatic tissues, tissue explants, or cells grown in culture.

In some embodiments, the sample is obtained from a subject with an in-born error of metabolism including maple syrup urine disease, phenylketonuria, organic acidemias, homocystinuria, tyrosinemia, and urea cycle disorders.

In some embodiments, the sample comprises cerebral spinal fluid, synovial fluid, lymph, peritoneal fluid, amniotic fluid, saliva, breast milk, gastric juice, bile, perspiration, tears, semen, vaginal secretions, breast milk, ascitic fluid, mucous, urine or pus.

In the methods disclosed herein, a sample may be processed to remove solid materials or other contaminants which could interfere with RP-HPLC. For example, spin filtering may be used to process a sample, preferably, without the need to dilute the sample or precipitate proteins or other materials with acid. In some embodiments, a 3K spin filter is selected to remove proteins by centrifugation. This method replaces the more common way of removing large proteins by acid precipitation. An advantage of this method over acid precipitation is it does not dilute the sample, thus increasing the single strength of each amino acid.

In some alternative embodiments, proteins are not removed from the sample prior to derivatization and application to the column, for example, samples that do not contain substantial amounts of proteins. In such an alternative embodiment, the sample may be applied to the column without protein removal or spin-filtering.

The method disclosed above comprises derivatizing a sample with O-Phthalaldehyde (OPA). Preferably, derivatization is performed on an RP-HPLC injector needle immediately before injection of the sample into the RP-HPLC column.

In the method disclosed in this application, a binary mobile phase may be applied to the RP-HPLC column. In one example of a binary mobile phase, the mobile phase comprises solution (A) and solution (B), where (A) comprises an aqueous mixture of sodium phosphate, sodium borate, and sodium azide and (B) comprises a mixture of acetonitrile, methanol and water. In some embodiments, a mobile phase, such as (A) may contain 10, 20, 30, 40, or 50-60% by volume of acetonitrile, 20, 30, 40, 50, 60, 70, 80-90% methanol, and 20, 30, 40, 50, 60, 70-80% water. A mobile phase such as (A) may contain 5, 10, 20, 30, 40, 50, 60, 70, 80, 90-100 mM sodium phosphate, 10, 20, 30, 40, 50, 60, 70, 80, 90-100 mM sodium borate, and 0.01 to 0.1 % by weight sodium azide. Sodium phosphate and sodium borate content may be selected based on a need to buffer a sample, and sodium azide content based on need to prevent bacterial growth. A preferred mixture for mobile phase (A) is 20 mM sodium phosphate and 20 mM sodium borate but concentrations can range from 5 to 100 mM of each. A small amount 2-10 mM of sodium azide is added to prevent bacterial growth. Likewise, a preferred mixture for mobile phase (B) is 45% of acetonitrile and 45% of methanol but a range of 30 to 60% of each may be used, added water to bring the mixture to 100%.

The methods disclosed above may be performed with an RP-HPLC run time of <20, 20, 25, 30, 35, 40, 45, 50, 60, or >60 mins. Advantageously, the run time is above 30 minutes or less.

In the methods disclosed herein, the fractions produced by the RP-HPLC run may be detected with ultraviolet (“UV”) light at a wavelength ranging from 200 to 400 nm, preferably at a wavelength of, or about 338 nm ± 1, 2, 5, 10, 15 or 20 nm.

Another aspect of the invention as disclosed herein is a method for simultaneous determination of ammonium and primary amino acids in a same sample comprising (a) removing solids from a sample, (b) derivatizing the sample obtained from (a) by contacting it with O-Phthalaldehyde (OPA), (c) injecting the derivatized sample from (b) into a RP-HPLC column to produce fractions, and (d) detecting RP-HPLC resolved fractions or components as they elute from the RP-HPLC column by ultraviolet illumination.

This simultaneous method may be performed using samples having volumes no more than <10, 15, 20, 25, 30, 35, 40, 45, 50, 60, 70, 80, 90, 100, or >100 µL, such as biological samples having volumes as small as 25ul to as much as 500 ul.

In some embodiments, the sample is obtained from a subject with an in-born error of metabolism including maple syrup urine disease, phenylketonuria, organic acidemias, homocystinuria, tyrosinemia, and urea cycle disorders.

In some embodiments, the sample comprises cerebral spinal fluid, synovial fluid, lymph, peritoneal fluid, amniotic fluid, saliva, breast milk, gastric juice, bile, perspiration, tears, semen, vaginal secretions, breast milk, ascitic fluid, mucous, urine or pus.

In the simultaneous method disclosed above, a sample may be processed to remove solid materials or other contaminants which could interfere with RP-HPLC. For example, spin filtering may be used to process a sample, preferably, without the need to dilute the sample or precipitate proteins or other materials with acid. In some embodiments, a spin filter is selected to remove proteins by ultrafiltration or by protein size exclusion.

In some alternative embodiments of this simultaneous method, proteins are not removed from the sample prior to derivatization and application to the column, for example, samples that do not contain substantial amounts of proteins. In such an alternative embodiment, the sample may be applied to the column without protein removal or spin-filtering.

The method disclosed above comprises derivatizing a sample with O-Phthalaldehyde (OPA). Preferably, derivatization is performed on an RT-HPLC injector needle immediately before injection of the sample into the RP-HPLC column.

In the simultaneous method disclosed above, a binary mobile phase may be applied to the RP-HPLC column. In one example of a binary mobile phase, the mobile phase comprises solution (A) and solution (B), where (A) comprises an aqueous mixture of sodium phosphate, sodium borate, and sodium azide and (B) comprises a mixture of acetonitrile, methanol and water. This method has an advantage of using the same mobile phase used for the amino acids determination for determining the level of ammonium concentration in the same run. However, using different mobile phases would force the user to do two separate assays.

The methods disclosed above may be performed with an RP-HPLC run time of <20, 20, 25, 30, 35, 40, 45, 50, 60, or >60 mins. Advantageously, the run time is above 30 minutes or less.

In the methods disclosed herein, the fractions produced by the RP-HPLC run may be detected with ultraviolet (“UV”) light at a wavelength ranging from 200 to 400 nm, preferably at a wavelength of, or about 338 nm ± 20 nm.

Another embodiment of the invention is directed to a method for calculating an intercellular volume of cells used to produce a cell lysate and detecting amino acid concentrations, comprising (a) producing a cell lysate, (b) adding a known concentration of a non-naturally occurring amino acid to the cell lysate to produce a sample for RP-HPLC, (c) derivatizing the sample obtained from (b) by contacting it with O-Phthalaldehyde (OPA), (d) injecting the derivatized sample from (c) into a RP-HPLC column to produce fractions, (e) detecting RP-HPLC resolved fractions or components as they elute from the RP-HPLC column by ultraviolet illumination; and (f) calculating an average intracellular volume of the cells used to produce the lysate by comparison of the concentration of the non-naturally occurring amino acid and the detected concentrations of one or more analytes with the concentration of the non-naturally occurring amino acid, thereby calculate an average cell volume of the cells used to produce the cell lysate.

In some embodiments of the method for calculating intracellular volume, the method may be performed using samples having no volumes more than <10, 15, 20, 25, 30, 35, 40, 45, 50, 60, 70, 80, 90, 100, or>100 µL, such as biological samples having volumes of 25 µL or less.

In some embodiments of this method the cells comprise red blood cells (RBCs). In other embodiments, the cells comprise leukocytes, cells from a somatic tissue, or cells from an ex vivo explant, or in vitro cell culture..

In the method disclosed above, a sample may be processed to remove solid materials or other contaminants which could interfere with RP-HPLC. For example, spin filtering may be used to process a sample, preferably, without the need to dilute the sample or precipitate proteins or other materials with acid. In some embodiments, a spin filter is selected to remove proteins by ultrafiltration or by protein size exclusion.

In some alternative embodiments of this simultaneous method, proteins are not removed from the sample prior to derivatization and application to the column, for example, samples that do not contain substantial amounts of proteins. In such an alternative embodiment, the sample may be applied to the column without protein removal or spin-filtering.

The method disclosed above comprises derivatizing a sample with O-Phthalaldehyde (OPA). Preferably, derivatization is performed on an RT-HPLC injector needle immediately before injection of the sample into the RP-HPLC column.

There are other compounds that have been used to derivatize amino acids. However, selection of OPA provides superior sensitivity and substitution of a different agent would alter retention times of the amino acids.

In the method disclosed above, a binary mobile phase may be applied to the RP-HPLC column. In one example of a binary mobile phase, the mobile phase comprises solution (A) and solution (B), where (A) comprises an aqueous mixture of sodium phosphate, sodium borate, and sodium azide and (B) comprises a mixture of acetonitrile, methanol and water. Content ranges for the mobile phases (A) and (B) are disclosed elsewhere herein.

The methods for determining cell volume disclosed above may be performed with an RP-HPLC run time of <20, 20, 25, 30, 35, 40, 45, 50, 60, or >60 mins. Advantageously, the run time is 30 minutes. Ranges for mobile phase concentrations and preferred mobile phases are described above.

Although the retention time is unique for each amino acid, the total run time is approximately 30 minutes. The equilibration time can vary from 10 to 30 minutes, which prepares the analytical column for the next injection.

In the methods for determining cell volume disclosed herein, the fractions produced by the RP-HPLC run may be detected with ultraviolet (“UV”) light at a wavelength ranging from 200 to 400 nm, preferably at a wavelength of, or about 338 nm ± 20 nm.

The foregoing paragraphs have been provided by way of general introduction and are not intended to limit the scope of the following claims. The described embodiments, together with further advantages, will be best understood by reference to the following detailed description taken in conjunction with the accompanying drawings.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings below.

FIGS. 1A and 1B respectively describe methionine and citrulline concentrations curves.

FIG. 2 shows a sample chromatogram from the method for detecting amino acids disclosed herein. 100 µM concentration of all amino acid except cystine, beta-alanine, and homocysteine which are 50 µM and our internal standard D-2-Aminobutyric acid (AABA) at 500 µM. FIGS. 1A, 1B and 2 pertain to the first method for detecting amino acids disclosed below.

FIG. 3 is similar to FIG. 2 and provides an example of the resolution of amino acid standards and with a retention time of 29.45 for ammonium.

FIG. 4 describes an ammonium linearity curve. FIGS. 3 and 4 pertain to the second method disclosed below which involves the simultaneous detection of amino acids and ammonium.

FIG. 5 is an example of the amino acids one is able to detect using the above method for analyzing amino acids from Part 3.

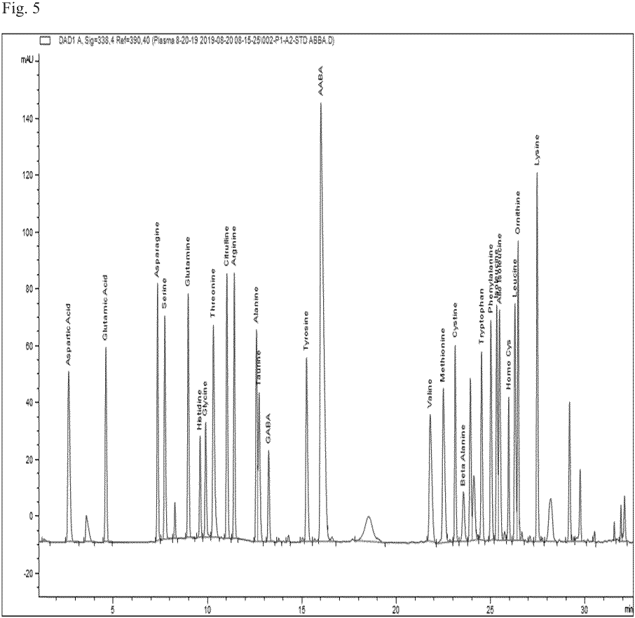

FIG. 6. Chromatograph of internal standard 1 mM AABA.

FIG. 7. Chromatograph of amino acids for 1 to 2 dilution

FIG. 8. HepG2 Cell Lysate Chromatograph. FIGS. 5-8 pertain to the third method disclosed below.

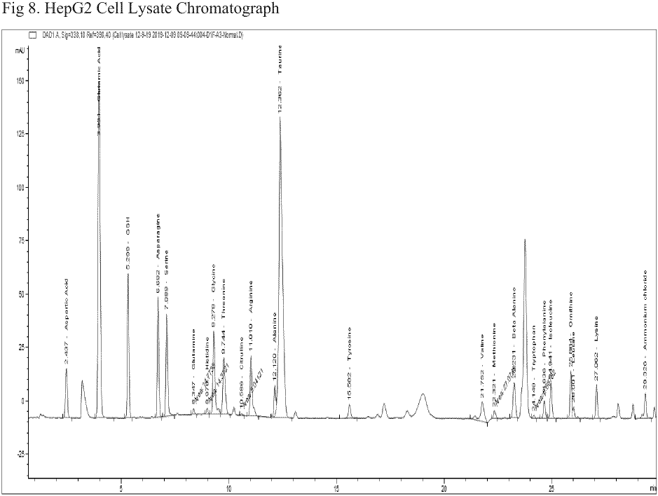

### DETAILED DESCRIPTION OF THE INVENTION

### Part 1

A rapid and easily deployed method for sensitive amino measurement in biological samples.

We have developed a fast and accurate method that uses a small volume of sample to determine over 25 of the typically reported amino acids in human plasma. Samples were prepped with a single step using a spin filter to remove proteins, avoiding the decreased sensitivity from dilution in acid precipitation. Using a reverse phase (RP) High Performance Liquid Chromatography (HPLC) system with O-Phthalaldehyde (OPA) as the pre-column derivatization reagent, and UV detection at 338 nm, the inventors did a direct comparison with the most common ion exchange/ninhydrin method used in clinical labs on the same plasma samples with 95% concurrence. With a sample preparation time of 30 minutes, utilizing less than 25 µ1 of sample and with a chromatography run of 30 minutes, this method can substantially increase workflow in both clinical and research laboratories using instruments widely available.

The measurement of amino acids in fluids is a basic analytic tool for the field of inborn errors of metabolism. Amino acid concentration levels in plasma are in the micromolar (uM) range and can also be found in most body fluids and tissues; . M. Akram, et al., Amino acids: A review article. JOURNAL OF MEDICINAL PLANTS RESEARCH, 5(17), 3997-4000. Their role in measurement is primarily in diagnosing and monitoring inborn errors of metabolism like, maple syrup urine disease, phenylketonuria, organic acidemias, homocystinuria, tyrosinemia, and urea cycle disorders; Blackburn Patrick R. et al., Maple syrup urine disease: mechanisms and management. THE APPLICATION OF CLINICAL GENETICS, 2017, 10, 57-66; Christodoulou John et al., Phenylketonuria: a review of current and future treatments. TRANSLATIONAL PEDIATRICS, 2015, 4(4), 314-317; Vaidyanathan Kannan et al., Organic Acidurias: An Updated Review. INDIAN JOURNAL OF CLINICAL BIOCHEMISTRY, 2011, 26(4), 319-325; Perry IJ., Homocysteine, hypertension, and stroke. JOURNAL OF HUMAN HYPERTENSION, 1999, 13, 289-293; Chinsky Jeffrey M. et al, Diagnosis and treatment of tyrosinemia type I: a US and Canadian consensus group review and recommendations. AMERICAN COLLEGE OF MEDICAL GENETICS AND GENOMICS. Advance online publication, 2017, 1-16; and Summar Marshall, Urea Cycle Disorders (UCD). NORD PHYSICIAN GUIDE TO THE UREA CYCLE DISORDERS (UCD) 2018.

Their importance can be summarized by the many different methods developed over the years for measuring these biomarkers; Walker V., et al., Quantitative methods for amino acid analysis in biological fluids. ANN CLIN BIOCHEM, 1995, 32: 28-57; Molnar-Perl Ibolya. Advancement in the derivatizations of the amino groups with the o-Phthalaldehyde-thiol and with the 9-fluorenylmethyloxycarbonyl chloride reagents. JOURNAL OF CHROMATOGRAPHY B, 879, 2011, 1241-1269; Csapo J., et al., Separation and determination of the amino acids by ion exchange column chromatography applying post column derivatization. ACTA UNIV. SAPIENTIAE, ALIMENTARIA, 1, 2008, 5 {29}; Kaspar H., et al., Automated GC-MS analysis of free amino acids in biological fluids. JOURNAL OF CHROMATOGRAPHY B, 870, 2008, 222-232; and Armstrong M., et al., Analysis of 25 underivatized amino acids in human plasma using ion-pairing reversed-phase liquid chromatography/time-of-flight mass spectrometry. RAPID COMMUN. MASS SPECTRUM, 2007, 21: 2717-2726.

The timely measurement of amino acids is highly impactful on the care of these patients; Burton Barbara. Inborn Errors of Metabolism in Infancy, A Guide to Diagnosis. PEDIATRICS, 1998, 102(6). With the widespread growth of newborn screening internationally, a rapid, small-volume, and reliable method of amino acid measurement on widely available standard lab equipment can benefit the field.

Currently the most common method of AA measurement utilizes ion-exchange chromatography with UV-colorimetric detection based on the chromophore formed when a primary amino acid reacts with ninhydrin; Rosen Hyman. A Modified Ninhydrin Colorimetric Analysis for Amino Acids. ARCHIVES OF BIOCHEMISTRY AND BIOPHYSICS, 1957, 67(1), 10-15; and Yemm E.W., et al., The Determination of Amino-Acids with Ninhydrin. THE ANALYST, 1955, 80(948), p. 209.

This reaction has been used for over 60 years with a high degree of confidence; however, a shortcoming of this method is the 2-to-3-hour runtimes per sample; see Hyman (1957), supra. In the last 20 years, other methods using HPLC, and LCMS have been published but not widely adopted. See Walker et al. supra; Molnar-Perl supra; Armstrong et al. supra; Hyman et al.; Yemm et al. supra; Fekkes Durk. State-of-the-art of high-performance liquid chromatographic analysis of amino acids in physiological samples. JOURNAL OF CHROMATOGRAPHY B, 1996, 682, 3-22; and Lookhart G, et al., High Performance Liquid Chromatography Analysis of Amino Acids at the Picomole Level. CEREAL CHEMISTRY 62(2):97-102.

We examined each step in the process of AA measurement and sought to optimize it for sample use, time, reproducibility, and equipment use. In this work, the specifics of the resulting method from this work are outlined, show results from biological samples, demonstrate the linearity of the method across a range of concentrations, measure the recovery using biological samples spiked with known amounts of several amino acids, and show the increase in sensitivity of a spin-filter preparation method over acid precipitation. In this method, all amino acid measurements were performed by reverse phase HPLC, using a two buffers gradient system (instead of the five buffer systems used commonly with most ion exchange systems) with a widely available C18 column; see Molna-Perl, supra. In some alternative embodiments of the methods disclosed herein, other types of columns may be used, such as C4, C8, phenyl, cyano, amino or silica type chromatographic materials.

Sample preparation was done through the use of an economical 3K centrifuge filter instead of an acid or alcohol precipitation. This filtration step avoids the formation of precipitates in the sample which can clog the analytical column. The filter has an added advantage of not diluting the sample, which improves the detection limits of the assay and allows less sample to be used.

The inventors consider that that this method may increase access to amino acid measurement in laboratories where standard HPLC equipment is available. This method can also decrease the required sample volume which is important for newborns and patients already having large volume blood draws. The reduction in analytical time may also provide more timely information for clinical decision making.

### Example 1

A rapid and easily deployed method for sensitive amino measurement in biological samples.

### Materials & Methods

Chemicals and Columns. The Infinity Lab Poroshell 120, 2.7 µm analytical column (C18) and the corresponding guard column, borate buffer and O-Phthalaldehyde (OPA) reagent were purchased from Agilent Technologies (Santa Clara, CA). HPLC grade Acetonitrile, methanol, and water were purchased from VWR International (Radnor, PA). Individual amino acids as well as an amino acid standard, and all other chemicals were purchased from Sigma Aldrich (St. Louis, MO). Argon gas was purchased from Roberts Oxygen Co (Rockville Md).

Equipment. The 1260 Infinity II LC System was purchased from Agilent Technologies (Santa Clara, CA). The inventors have a 40 uL syringe and sample loop for their HPLC system. The Centrifuge 5417c was purchased from Eppendorf (Hamburg, Germany). The vortex mixer was purchased from BioExpress (Kaysville, UT). The 3 k centrifuge filters (VWR Spin filter 3k, 82031-346) were purchased from VWR international.

Chromatographic conditions. A binary mobile phase consisting of solution A, 20 mM sodium phosphate (dibasic), 20 mM sodium borate, and 5 mM sodium azide, adjusted pH to 7.2 and solution B, a mixture of 45% acetonitrile, 45% methanol, and 10% water were used. Programming for the chromatographic run starts with 100% of solvent A, 0% solvent B and over a 30-minute time course reduces A to 0% and B to 100% as shown in Table 1. Then the column will equilibrate back to 100% solvent A over 10 minutes. The column temperature was held constant at 40° C. while the sample tray was maintained at 4° C. UV detection was performed at 338 nm.

TABLE 1

Chromatographic Program

| Time (min) | Solvent A (%) | Solvent B (%) |
|---|---|---|
| 0 | 100 | 0 |
| 6 | 90 | 10 |
| 13.5 | 80 | 20 |
| 18 | 80 | 20 |
| 25 | 60 | 40 |
| 26 | 60 | 40 |
| 29 | 40 | 60 |
| 30 | 0 | 100 |
| 34 | 0 | 100 |
| 35 | 100 | 0 |
| 40 | 100 | 0 |

Mobile Phase Concentration Over Time (includes column equilibration min 30-40)

Derivatization and Injection. OPA purchased from Agilent was used to derivatize the primary amino group. OPA has been tested before but not commonly used since its formed derivatives are only stable for 2-6 hours; Armstrong M., et al., Analysis of 25 underivatized amino acids in human plasma using ion-pairing reversed-phase liquid chromatography/time-of-flight mass spectrometry. RAPID COMMUN. MASS SPECTRUM. 2007; 21: 2717-2726.

The inventors have taken advantage of the advent of programable injectors, and derivatize samples in the injector’s needle immediately before column injection. This allows one the benefit of the stronger absorbance of the OPA reagent in the 30-minute run time. The injector was programmed to draw 16 µL of borate buffer pH 10.4 then 4 µL of the sample and mix with 10 µL of air, then draw 3 µL from OPA reagent, mix with 10 µL of air, wait 2 minutes, and inject 23 µL. If the OPA solution is stored under Argon in a sample vial, it will be stable for up to 2 weeks. The program steps are outlined in Table 2.

TABLE 2

Programming steps of RD-HP{LC run specific for Agilent model 1260.
Injector Program Primary AA on Agilent model 1260

| | |
|---|---|
| Draw | Draw 16 µL of Borate buffer with default speed using default offset. |
| Draw | Draw 4 µL from sample with default speed using default offset. |
| Mix | Mix 10 µL from air with default speed 5 times. |
| Wait | Wait 0.2 minutes. |
| Draw | Draw 3 µL of OPA with default speed using default offset. |
| Mix | Mix 10 µL from air with default speed 7 times. |
| Wait | Wait 2 minutes. |
| Inject | Inject. |
| Wait | Wait 0.5 minutes. |
| Valve | Switch valve to “bypass”. |

Biological sample: As part of this quality improvement effort, human samples were collected in standard EDTA tubes for routine amino acid analysis and frozen residuals used for our analysis without identifiers. An aliquot of sample was centrifuged at 1200 g for 10 minutes. Plasma and red blood cells were collected in 100 µL aliquots and stored in -80° C. Personnel were blinded to the origin of the sample and comparison results were provided without patient ID or clinical information.

### Biological Sample Preparation

Filter Sample Preparation: A plasma (or other liquid sample) aliquot is transferred to a pre-wet 3K centrifugal filter and centrifuged for 20 minutes at 9000 g. The liquid is collected and transferred to vials for injection on the HPLC. The 3K filter is a polypropylene 1.7 ml sample tube with a PES membrane insert which traps structures greater than 3000 MW to remove excess proteins which could clog HPLC column and filter. The filter has a 5 ul hold back volume which requires a pre-wet of the filter before use. One passes 50 ul of PBS and discards the eluent before adding any sample.

Acid Precipitation Sample Preparation: The method used in most clinical labs uses acid precipitation. To a 50 µL aliquot of plasma add 100 µL of 0.15 M sulfosalicylic acid, and place on ice for 30 minutes to allow the proteins to precipitate before centrifuging for 10 minutes at 14000 g. Supernatant is collected and transferred to vials for injection on HPLC.

Amino Acid standards solutions: 5 mM stocks of the individual amino acids were prepared separately in 0.1 N hydrochloric acid and stored at 4° C. To check linearity and reproducibility, concentrations of 5-200 µM were made by diluting stocks with PBS. Standard concentrations were prepared fresh on the day of analysis

### Results and Discussion

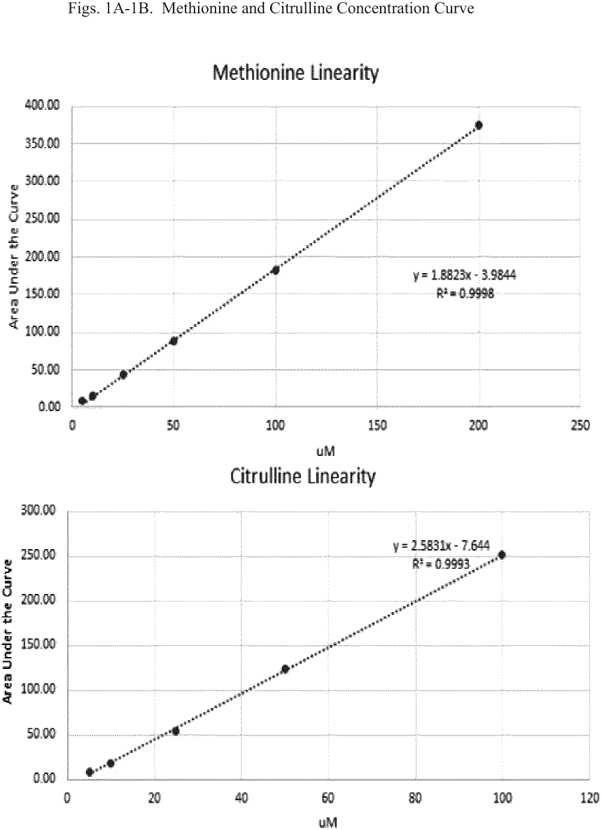

Standard curves over a range of concentration: The inventors show, as examples of linearity of measurement, two amino acids over a concentration range of 5 to 200 uM and 5 to 100 uM in FIGS. 1a and 1b. These standards were done in triplicates for each concentration, and it should be noted the r² values of 0.98 or higher are typical for all amino acids (data available).

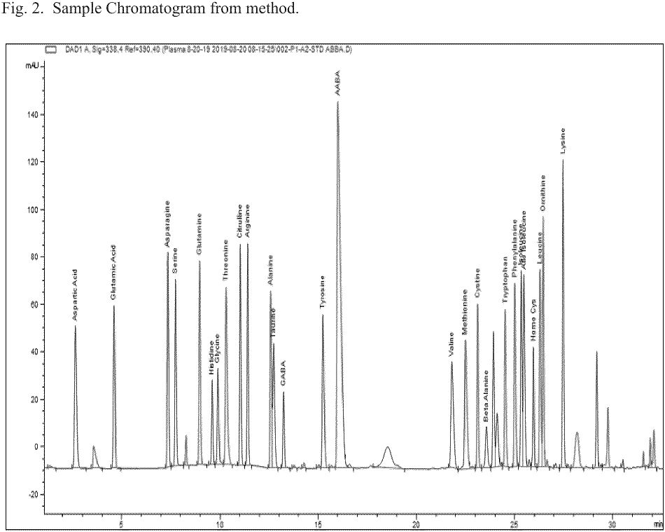

Amino Acids Chromatograph: Each amino acid was injected independently to resolve its retention time, and if needed the chromatic program was adjusted to avoid co-migrating of amino acids in order to achieve near baseline resolution. FIG. 2 shows a sample of our amino acid standard run under our method’s conditions.

Standard curves over a range of concentration: The inventors show, as examples of linearity of measurement, two amino acids over a concentration range of 5 to 200 uM and 5 to 100 uM in FIGS. 1a and 1b. These standards were done in triplicates for each concentration, and it should be noted the r² values of 0.98 or higher are typical for all amino acids.

Amino Acids Chromatograph: Each amino acid was injected independently to resolve its retention time, and if needed the chromatic program was adjusted to avoid co-migrating of amino acids in order to achieve near baseline resolution. FIG. 2 shows a sample of our amino acid standard run under our method’s conditions.

Comparing 3K filter results against the acid precipitation method. In comparing the methods of using a 3K centrifugal filter for protein removal to the more common method of acid precipitation used in plasma preparation the inventors compared a split sample with 3 repeats for each method. The results are shown in Table 3.

TABLE 3

| Amino Acid | 3K Filter Avg. pM | Acid Precipitation Avg. µM | Ratio Acid Precip. to 3K Filter |
|---|---|---|---|
| Glutamic Acid | 87 | 71 | 1.23 |
| Asparagine | 54 | 47 | 1.15 |
| Serine | 97 | 85 | 1.14 |
| Glutamine | 602 | 554 | 1.08 |
| Histidine | 55 | 48 | 1.13 |
| Glycine | 233 | 238 | 0.98 |
| Threonine | 85 | 77 | 1.11 |
| Citrulline | 13 | 12 | 1.11 |
| Arginine | 75 | 72 | 1.03 |
| Alanine | 409 | 335 | 1.22 |
| Tyrosine | 59 | 55 | 1.08 |
| Valine | 218 | 200 | 1.09 |
| Methionine | 18 | 16 | 1.07 |
| Cystine | 144 | 130 | 1.11 |
| Beta alanine | 41 | 37 | 1.11 |
| Tryptophan | 6 | 44 | 0.14 |
| Isoleucine | 44 | 45 | 0.99 |
| Phenylalanine | 68 | 63 | 1.09 |
| Leucine | 124 | 115 | 1.08 |
| ornithine | 79 | 73 | 1.08 |
| Lysine | 173 | 165 | 1.05 |
| Ammonium | 12 | 17 | 0.71 |

Comparison of detected concentrations between preparation with a 3K filter and acid precipitation. Three repeats for each method were performed.

The ratio of the two signals confirms that using the filters increases the signal strength of most amino acids. This is probably the result of increased sensitivity by not diluting the sample and avoiding loss through precipitation or acid modification.

The filter processed tryptophan is much lower than the acid precipitation. The filter was tested for tryptophan retention and was negative. The lower levels are most likely a result of reported binding of tryptophan to albumin which would be released by acid precipitation; McMenamy Rapier, Oncely J. L. The Specific Binding of L-Tryptophan to Serum Albumin. JOURNAL OF BIOCHEMISTRY (1958) 233:1436-1447.

Glutamate and alanine elevations in the non-acid precipitated sample suggesting an effect of the acid on the molecules.

Recovery study of spiked amino acids: A single plasma sample was divided into 10 equal aliquots. As described, the samples were spun using the 3K filter. To five of these samples 50 µM each of arginine, tyrosine, methionine, and isoleucine was added and none to the other 5. Samples were derivatized and run as described. Table 4 compares the amino acids in the 5 plasma aliquots from each group. Good recovery of the 50 uM was observed in the spiked aliquots (94-110%).

TABLE 4

Recovery of Spiked 50 µMAmino Acids using one plasma sample split into 10 aliquots with amino acids added to each aliquot before processing.

| Amino Acid Added | Unspiked Plasma µM± SD (n=5) | Spiked Plasma µM± SD (n=5) | Average Difference µM ± SD (Recovery) | % Recovery Average |
|---|---|---|---|---|
| Arginine | 74 ± 2.7 | 123 ± 6.4 | 50 ± 3.9 | 100% |
| Tyrosine | 67 ± 2.7 | 118 ± 5.6 | 51 ± 3.7 | 102% |
| Methionine | 15 ± 1.3 | 61 ± 4.1 | 47 ± 3.2 | 94% |
| Isoleucine | 71 ± 5.2 | 126 ± 8.2 | 55 ± 5.5 | 110% |

Comparing Clinical Laboratory Results to Our Protocol. Using ten de-identified 1-day old frozen discard samples from the clinical laboratory, the inventors compared concentration measurements by our method and the normal clinical laboratory method. The clinical laboratory uses a standard ion-exchange chromatography method with ninhydrin derivatization (average run time 3 hours). Table 5 shows the averages for the amino acid measures and the ratios between the methods (our method vs clinical lab). Agreement is close between the samples and except for tryptophan and glutamic acid which was expected from the filter vs acid precipitation measurements. Of note also is that glutamic acid had the highest patient to patient variability of the amino acids.

TABLE 5

| Amino Acid | A. Average Conc. Our Method µM | B. Average Conc. Clinical Laboratory Method µM | Ratio A/B |
|---|---|---|---|
| Beta alanine | 0 | 0 | |
| Homocystine | 0 | 0 | |
| L-allo- isoleucine | 0 | 0 | |
| Aspartic Acid | 3.2 | 3.6 | 0.89 |
| Tryptophan | 41.8 | 4.5 | 9.29 |
| Methionine | 23.1 | 20.2 | 1.14 |
| Cystine | 35.3 | 32.1 | 1.10 |
| Phenylalanine | 46.6 | 42 | 1.11 |
| Citrulline | 58.1 | 51.9 | 1.12 |
| Asparagine | 54.2 | 54.3 | 1.00 |
| Isoleucine | 50.1 | 54.6 | 0.92 |
| Taurine | 57.2 | 58.6 | 0.98 |
| Glutamic Acid | 41.9 | 59.2 | 0.71 |
| Tyrosine | 71.1 | 63.6 | 1.12 |
| Arginine | 64.9 | 65.7 | 0.99 |
| Ornithine | 61.3 | 70 | 0.88 |
| Histidine | 73.8 | 70.1 | 1.05 |
| Threonine | 100.1 | 83.3 | 1.20 |
| Leucine | 95.5 | 90.4 | 1.06 |
| Lysine | 128.3 | 120.7 | 1.06 |
| Serine | 132.7 | 129.3 | 1.03 |
| Valine | 190.4 | 171.2 | 1.11 |
| Alanine | 320.8 | 319.8 | 1.00 |
| Glycine | 359.5 | 379.1 | 0.95 |
| Glutamine | 521.8 | 514.2 | 1.01 |

Comparison of 10 De-Identified Split Patient Samples between the Clinical Laboratory and our OPA method. *⁺ The expected differences of Tryptophan and Glutamate are due to differences in preparation. Samples are sorted by average concentration from lowest to highest.

As disclosed herein, the inventors have shown that one can quantify amino acid levels in plasma via an RP HPLC method of 30 minutes run time followed by 10 minutes for wash and equilibration. This method uses 25 µL (or less) of sample with a preparation time of under 30 minutes. A complete range of amino acid compounds detected by current clinical measurement can be seen. This method uses less volume and the inventors have consistently achieved reproducible results with 10 µL of sample (data available). While the preparation time is comparable to current methods, the run time is only 15-20% of the current standard methods. The instrumentation and materials for this method are more cost-effective than traditional ion-exchange systems and widely available in most clinical labs. The use of filtration improves the sensitivity without the dilution from acid precipitation allowing more accuracy at lower concentrations of amino acids. Furthermore, our data suggests that a number of amino acid concentrations are affected by acid precipitation such as the decreases in alanine and glutamate noted. It is well known that tryptophan binds to albumin and this difference is reflected in our results which measures only unbound or free tryptophan; McMenamy Rapier, Oncely J. L. The Specific Binding of L-Tryptophan to Serum Albumin. JOURNAL OF BIOCHEMISTRY (1958) 233:1436-1447. The consistent recovery of added amino acids shows the reliability and robustness of the assay.

As the number of patients diagnosed with inborn errors of amino acid metabolism has increased globally the access to rapid testing has become more important. Rapid turnaround of results can be critical to patient management and therapeutic decision making for patients in crisis and for routine care. The relative cost-effectiveness of this method can either increase the capacity of existing laboratories or create capacity in laboratories where it doesn’t exist. In regions where there are resource limits, this can improve diagnostic access and outcome for patients and their families. In the research sphere, this lower cost method can increase throughput and provide broader sample analytics. The authors hope this method will expand access to amino acid measurements in clinical and research environments, leading to more rapid diagnosis of patients at a lower cost.

### Part 2

An assay for the simultaneous determination of ammonium and most primary amino acids using reverse phase (RP) High Performance Liquid Chromatography (HPLC) in plasma.

By using O-Phthalaldehyde (OPA) as the pre-column derivatization reagent, and at 338 nm for UV detection, the inventors have validated our assay for linearity, accuracy, and precision over a working range from 0-2000 uM.

Ammonia is an important source of nitrogen but can be life threatening in high concentrations a condition called hyperammonemia. Auron Ari, et al., Hyperammonemia in review: pathophysiology, diagnosis, and treatment. PEDIATR NEPHROL, 2011. Hyperammonemia is mostly due thru a urea cycle disorder, UCD. Machado Marcel, et al., Hyperammonemia due to urea cycle disorders: a potentially fatal condition in the intensive care setting. J OF INTENSIVE CARE, 2014, 2:22; Summar, Marshall, et al., Urea Cycle Disorders Overview. GENE REVIEWS 2003, 1-15. Much has been published in the study and management of this disorder. Ways have been developed to measure Ammonia in blood for as early as 1925. Murray Margaret. The estimation of ammonia and urea in blood and urine. THE PHYSIOLOGY DEPARTMENT OF BEDFORD COLLEGE UNIV OF LONDON, 1925, 294-299. Other methods include colorimetric/fluorometric reactions, enzymatic methods, and gas sensing electrodes. Ringuet, Stephanie, et al., A suite of microplate reader-based colorimetric methods to quantify ammonium, nitrate, orthophosphate, and silicate concentrations for aquatic nutrient monitoring. JOURNAL OF ENVIRONMENTAL MONITORING, 2011, 370-376; Pasha, Qadar, et al., A rapid method for plasma ammonia estimations using an indigenously purified enzyme. INDIAN JOURNAL OF CLINICAL BIOCHEMISTRY, 2000, 15(1), 29-35; Ayyub Omar, et al., Simple and inexpensive quantification of ammonia in whole blood. MOLECULAR GENETICS AND METABOLISM, 2015, 115, 95-100. Ammonium is most commonly measured in blood, water, and even breathe. Batsotti Robert. Measurement of ammonia in blood. J PEDIATR. 200, 138:S11-20; Park Gaeun, et al., Improvement of the ammonia analysis by the phenate method in water and wastewater. BULL KOREAN CHEM. SOC, 2009, 30(9), 2032-2038; and Spacek Lisa, et al., Repeated measures of blood and breathe ammonia in response to control, moderate and high protein dose in healthy men. SCIENTIFIC REPORTS, 2018 8:2554.

The amount of ammonia in collected blood, urine, saliva, or other biological fluid samples can be affected by several mechanisms that may lead to erroneous ammonia concentration determinations. These effects can be minimized by proper sample storage and handling. The ammonia content of freshly drawn blood rises rapidly on standing because of the deamination of labile amides such as glutamine; at room temperature, the ammonia content can increase by a factor of two or three in several hours. Henry RJ, Non-protein nitrogenous constituents. CLINICAL CHEMISTRY PRINCIPLES AND TECHNICS, Harper and Row 1964, 325-331. Therefore, it is important to both keep the specimen cold (on ice) and perform the analysis as soon as possible. If the sample cannot be analyzed quickly, it may be frozen (-20° C.). The ammonia content of iced (4° C.) blood samples remains constant for up to 60 minutes, whereas the ammonia content of frozen (-20° C.) blood samples remains constant for several days. Huizengo JR, et al., Determination of ammonia in biological fluids. ANAL CLINICAL BIOCHEM, 1994, 31(6) 529-543. For blood samples collected from a healthy person (and stored on ice), the ammonia content should be measured within 30-60 minutes of collection. For persons suspected of suffering from liver disease, however, the blood samples should be analyzed within 15 minutes. See Huizengo, supra This more rapid assessment is necessary because some liver diseases result in high levels of γ-glutamyl transferase, an enzyme that hydrolyzes glutamine; the enzyme’s activity will increase the concentration of ammonia in the sample to levels higher than was present at the time of collection. See Huizengo, supra.

### Example 2

A Method for Simultaneously Measuring Ammonium and Amino Acids in Plasma using an RP-HPLC

### Materials & Methods

Chemicals. The Infinity Lab Poroshell 120, 2.7 µm analytical column and the corresponding guard column, borate buffer and OPA reagent were purchased from Agilent Technologies (Santa Clara, CA). HPLC grade Acetonitrile, methanol, and water were purchased from VWR International (Radnor, PA). Individual amino acids as well as an amino acid standard, and all other chemicals were purchased from Sigma Aldrich (St. Louis, MO). Argon gas was purchased from Roberts Oxygen Co (Rockville Md).

Equipment. The 1260 Infinity II LC System was purchased from Agilent Technologies (Santa Clara, CA). The Centrifuge 5417c was purchased from Eppendorf (Hamburg, Germany). The vortex mixer was purchased from BioExpress (Kaysville, UT). The 3k centrifuge filters were purchased from VWR international.

Chromatographic conditions. A binary mobile phase consisting of solution A, 20 mM sodium phosphate (dibasic), 20 mM sodium borate, and 5 mM sodium azide, adjusted pH to 7.2 and solution B, a mixture of 45% acetonitrile, 45% methanol, and 10% water were used. The chromatographic run starts with 100% of solvent slowly changing to 90% A in 6 minutes, and 80% A at 13.5 minutes. Then the ratio is held until 18 minutes, when it shifts to 60/40 ratio towards 25 minutes, and held at this ratio until 26 minutes. Then it would switch towards 40/60 ratio at 29 minutes and go to 100% solvent B at 30 minutes. Between 30 and 34 minutes is a wash using solvent B, and then the column will equilibrate by holding at 100% solvent A until 40 minutes. The column temperature was held constant at 40° C. while the sample tray was maintained at 4⁰C. UV detection was performed at 338 nm.

TABLE 1

Mobile Phase Concentration Over Time

| Time (min) | Solvent A (%) | Solvent B (%) |
|---|---|---|
| 0 | 100 | 0 |
| 6 | 90 | 10 |
| 13.5 | 80 | 20 |
| 18 | 80 | 20 |
| 25 | 60 | 40 |
| 26 | 60 | 40 |
| 29 | 40 | 60 |
| 30 | 0 | 100 |
| 34 | 0 | 100 |
| 35 | 100 | 0 |
| 40 | 100 | 0 |

Derivatization and Injection. The inventors used OPA purchased from Agilent to derivatize the primary amino group. OPA has been used before but because the derivatives formed are only stable for 2-6 hours, which is not practical to use (18). Because now the inventors have the ability to program the injector, one can derivatize samples in the injector’s needle before injection. The injector was programmed to draw 15 µL of borate buffer pH 10.4 then 3 µL of the sample and mix with 10 µL of air, then draw 2 µL from OPA reagent, mix with 10 µL of air, wait 2 minutes, and inject 20 µL. It is important to note that if OPA solution is stored under Argon in a sample vial, it would be stable for 2 weeks.

### Injector Program Primary AA on 1260

Draw Draw 15 µL of Borate buffer with default speed using default offset.

Draw Draw 3 µL from sample with default speed using default offset.

Mix Mix 10 µL from air with default speed 5 times.

Wait Wait 0.2 minutes.

Draw Draw 2 µL of OPA with default speed using default offset.

Mix Mix 10 µL from air with default speed 7 times.

Wait Wait 2 minutes.

Inject Inject.

Wait Wait 0.5 minutes.

Valve Switch valve to “bypass”.

Amino Acid standards solutions: 5 mM stocks of the individual amino acids were prepared separately in 10 ml 0.1 N hydrochloric acid and stored at 4° C. These stock solutions have been stable for up to 6 months. To check linearity and reproducibility, concentrations of 0-2000 µM were made by diluting stocks with PBS. Standards were prepared fresh on the day of analysis.

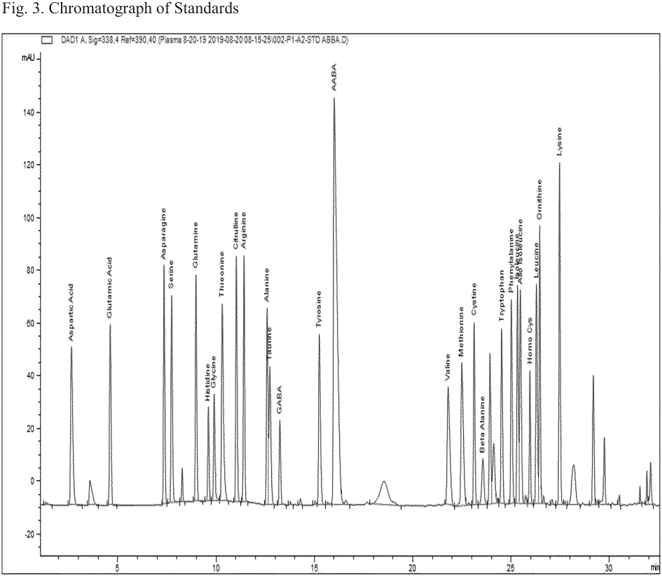

Mobile phase preparation: Mobile phase A consist of 20 mM sodium borate, 20 mM sodium phosphate (dibasic) and 5 mM sodium azide. It is prepared by dissolving 3.3 grams of dibasic sodium phosphate, 7.6 grams of sodium borate and 325 mg of sodium azide in 1 liter of water then pH to 7.2 with phosphoric acid. Mobile phase B is mixture of 45% acetonitrile, 45% methanol, and 10% water. FIG. 3 is an example of our amino acid standards with the retention time of 29.45 for ammonium.

Precipitating Reagent Solution (SSA). This is 0.15 M sulfosalicylic acid buffer, pH 2.0. To make, dissolve 15 g sulfosalicylic acid stock in approximately 395 mL DI water in a 400 mL volumetric flask. Adjust pH to 2.0 then QS to 400 mL with DI water. Store refrigerated (2 -8° C.) in amber glass vials with Teflon-lined caps. Stable 6 months.

Whole Blood preparation: Whole blood was collected from human volunteers. After the collection, the blood was centrifuged at 1200 g for 10 minutes. Plasma and red blood cells were collected in 100 µL aliquots and stored in -80° C. for later analysis.

Plasma Sample preparation: To 10 uL of plasma add 20 uL of SSA and let sit on ice for 30 minutes before centrifuging at 14000 rpm for 5 minutes. Transfer top liquid to an insert in a sample vial for injection.

### Results

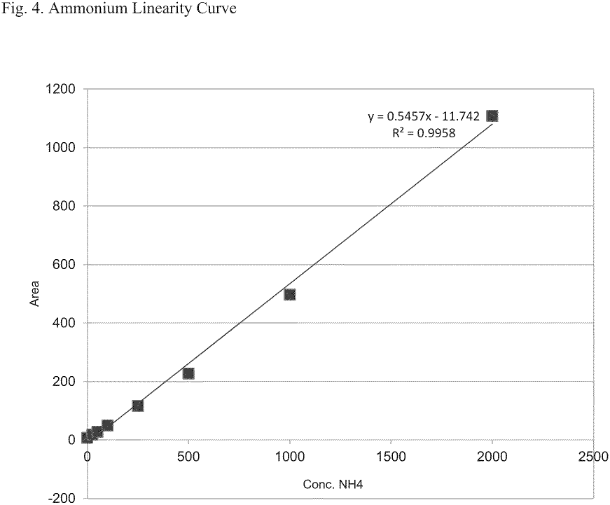

FIG. 4 shows a linearity curve from 0 to 2000 uM of ammonium standards.

Next, theinventors did a series of plasma samples where they tried two different dilutions with SSA a 1 to 2 and a 1 to 3 dilution on baseline and multiple spiking amounts to test for recovery and consistency. Results of the recovery study are shown in Table 2 below.

TABLE 2

Recovery Study

| | Concentration adjusted for 1 to 2 dilution | concertration adjusted for background | percent recovery | Concentration adjusted for 1 to 3 dilution | concertration adjusted for background | percent recovery |
|---|---|---|---|---|---|---|
| 0 or background | | 56.86 | | | 81.41 | |
| 50 | 90.06 | 33.20 | 66.39% | 133.25 | 51.83 | 103.67% |
| 100 | 142.20 | 85.34 | 85.34% | 172.77 | 91.36 | 91.36% |
| 250 | 289.33 | 232.47 | 92.99% | 315.64 | 234.23 | 93.69% |
| 500 | 562.50 | 505.64 | 101.13% | 554.79 | 473.37 | 94.67% |
| 1000 | 1,087.58.00 | 1030.71 | 103.07% | 1020.58 | 939.17 | 93.92% |
| 2000 | 2,166.76.00 | 2109.90 | 105.49% | 2135.77 | 2054.36 | 102.72% |

This work has been repeated this with identical results. Although not a great difference in the different dilutions with the acid, the inventors consider the 1 to 3 dilution gave slightly better numbers.

It is interesting to note several papers showing ammonium levels increasing with time (14). Fresh blood draws from several volunteers where each sample was centrifuged, and the plasma removed and placed on ice. Then aliquots were taken from these samples immediately for a baseline or T=0, and additional aliquots from the same sample after 1, 2, and 4 hours to measure the ammonium levels. Table 3 shows the results and includes a couple of amino acid levels over this period of time to show only the ammonium seems to change as has been reported.

TABLE 3

Ammonium levels uM concentration

| | Patient 1 | Patient 2 | Patient 3 | Patient 4 |
|---|---|---|---|---|
| Time 0 hr | 34.93 | 40.31 | 23.01 | 43.86 |
| Time 1 hr | 46.67 | 53.35 | 41.81 | 61.53 |
| Time 2 hr | 53.23 | 54.54 | 47.28 | 73.75 |
| Time 4 hr | 55.3 | 58.13 | 47.81 | 62.39 |

Table 3 above shows changes in Plasma Ammonium over a 4 hour time course

Tables 4 and 5 show during these times other amino acids, Ornithine and Glutamine as examples, are relative constant.

TABLE 4

Ornithine levels over a 4 hour time course
Ornithine levels uM concentration

| | Patient 1 | Patient 2 | Patient 3 | Patient 4 |
|---|---|---|---|---|
| Time 0 hr | 55.78 | 76.53 | 45.14 | 99.53 |
| Time 1 hr | 54.55 | 80.97 | 45.65 | 99.85 |
| Time 2 hr | 54.98 | 77.89 | 43.54 | 97.1 |
| Time 4 hr | 54.43 | 70.04 | 42.26 | 87.59 |

TABLE 5

Glutamine levels over a 4 hour time course
Glutamine levels uM concentration

| | | | |
|---|---|---|---|
| Patient 1 | Patient 2 | Patient 3 | Patient 4 |

| | | | | |
|---|---|---|---|---|
| Time 0 hr | 337.85 | 327.33 | 308.8 | 380.51 |
| Time 1 hr | 331.38 | 341.45 | 319.8 | 375.44 |
| Time 2 hr | 328.5 | 334.53 | 308.09 | 373.66 |
| Time 4 hr | 334.65 | 303.19 | 301.51 | 338.28 |

Detailed Catalog of Materials Used in Example 3

| | Part Number | Company |
|---|---|---|
| AA standard | | |
| Amino Acid Standard | AAS18-10ML | Sigma |
| L-Amino Acids | LAA21-KT | Sigma |
| L-Glutathione oxidized | G4376-5G | Sigma |
| L-Glutathione reduced | G6529-5G | Sigma |
| L-Allo-Isoleucine | 18754-100MG | Sigma |
| Beta Alanine | PHR1349-1G | Sigma |
| γ-Aminobutyric acid | 03835-250MG | Sigma |
| L-Omithine monohydrochloride | O6503-25G | Sigma |
| L-Homocystine | H6010-100MG | Sigma |
| L-Citrulline | C7629-100G | Sigma |
| Taurine | 166541000 | ACROS |
| D-2-Aminobutyric acid (AABA) | 116122-5G | Sigma |
| Derivatization Reagent | | |
| O-phthaldialdehyde Reagent Solution (OPA) | 5061-3335 | Agilent |
| Borate Buffer | 5061-3339 | Agilent |
| Column | | |
| Poroshell HPH-C18, 3.0x100 mm 2.7um | 695975-502 | Agilent |
| UHPLC Grd, Poroshell HPH-C18, 3.0 mm | 823750-928 | Agilent |
| Sodium phosphate diabasic | S9763-1KG | Sigma |
| Sodium azide | S8032-100G | Sigma |
| Sodium tetraborate decahydrate | S9640-500G | Sigma |
| Buffer 2 | | |
| Acetonitrile HPLC Grade | BDH83639.400 | VWR |
| Methanol HPLC Grade | BDH20864.400 | VWR |
| Water HPLC Grade | BDH23595.400 | VWR |
| Hydrochloric acid 5.0N | BDH7419-1 | VWR |
| Phosphoric Acid | BDH3104-2.5PLC | VWR |
| VWR Spin filter 3k | 82031-346 | VWR |
| Sulfosalicylic Acid S2130-100 Sigma Cap screw blue | 97052-794 | VWR |
| Insert MS plastic spring | 97051-410 | VWR |
| Vials (Glass) | 5182-0714 | Agilent |
| Vials (Amber borosilicate) | 5182-0716 | Agilent |

The inventors have demonstrated the strength of the disclosed method and how it combines a prior method developed by the inventors for primary amino acids with an added ability to simultaneously. The data provided shows the method’s linearity, stability, and sensitivity, and recovery. Simultaneous measurement of these types of biomarkers in a single assay provides a simple and convenient method application for clinical diagnostics.

### Part 3

An assay using reverse phase (RP) High Performance Liquid Chromatography (HPLC) to calculate intercellular volumes in cell lysate, red blood cells (RBC), and tissue.

By the addition of a known volume and concentration of an internal standard, which is a non-natural occurring amino acid, one can use the difference in concentration of this standard caused by its dilution due to the unknows volume on the concentration of the internal standard and calculate the intercellular volume. This will now give one the ability to calculate the concentration in these samples and not rely on normalizing to protein amounts and compare directly to plasma concentrations.

Given the problem for analyzing red blood cells sample when often they have clotted or frozen tissue or cell lysates after being stored at -80 for weeks seems daunting. These samples are often not aliquoted by a known volume or weighed before storage. One can measure the concentration of amino acids in these samples but without a known intracellular volume one cannot calculate a true concentration and are forced to normalize by protein amounts and there are multiply methods for measuring protein, Bradford, Lowry, BCA, and others. Bradford, MM.. A rapid and sensitive method for the quantitation of microgram quantities of protein utilizing the principle of protein-dye binding. ANALYTICAL BIOCHEMISTRY. 1976, 72, 248-254; Lowry, O.H., et al., Protein measurement with folin phenol reagent. JOURNAL OF BIOLOGICAL CHEMISTRY, 1951, 193, 265-75; Smith, P.K, et al., Measurement of protein using bicinchoninic acid. ANALYTICAL BIOCHEMISTRY, 1987, 150, 76-85; and Krohn, R.I., The Colorimetric Determination of Total Protein, Current Protocols in Food Analytical Chemistry, B1.1.1-B1.1.27, John Wiley & Sons, Inc., 2001.

Other considerations when homogenizing any sample. One wants to use as little as possible so not to dilute the signal but as some point is using too little solution may be bad. Is it possible to calculate both volume and concentration when both are unknown? Simply put how does one solve for V₂ and C₂ when both are unknown? A method to calculate this intracellular volume first is disclosed, then use of this volume to calculate the amino acid concentration in these types of matrices.

### Example 3

A RP-HPLC Method to determine Glutathione concentrations in a cell lysate and red blood cell samples, by first determining the intercellular volume.

### Materials & Methods

Chemicals: Borate Buffer and OPA reagent were purchased from Agilent Technologies (Santa Clara, CA). HPLC grade Acetonitrile, methanol, and water were purchased from VWR International (Radnor, PA). GSH, GSSG, and all other chemicals used for the amino acid standard were purchased from Sigma Aldrich (St. Louis, MO) including our internal standard D-2-Aminobutyric acid (AABA).

Equipment: The 1260 Infinity II LC System was purchased from Agilent Technologies (Santa Clara, CA). The Centrifuge 5417c was purchased from Eppendorf (Hamburg, Germany). The vortex mixer was purchased from BioExpress (Kaysville, UT).

Amino Acid standards solutions: 5 mM stocks of the individual amino acids were prepared separately in 10 ml 0.1 N hydrochloric acid and stored for six months at 4C. To check linearity and reproducibility concentrations of 50-250 µM were made by diluting stocks with PBS. Standard concentrations were prepared fresh from these stocks on the day of analysis

Chromatographic conditions: A binary mobile phase consisting of solution A, 20 mM sodium phosphate (dibasic), 20 mM sodium borate, and 5 mM sodium azide, adjusted pH to7.2 and solution B, a mixture of 45% acetonitrile, 45% methanol, and 10% water used. The chromatographic run is started with 100% solution A, then a gradient change to 80% A over the next 12 min, hold at 80% solution A and 20% solution B until 18 min then a 60/40 % gradient of A/B is obtained at 25 min, hold for one minute, than a 40/60 % gradient of solution A/B by 29 min, then 0/100 % solvent A/B change in one minute and hold this concentration until 34 min, lastly go to 100% A in one minute and hold a column equilibration period of 100% solution A for 5 min. The column temperature was held constant at 40° C. while the sample tray was maintained at 4° C. UV detection was performed at 338 nm.

The injector is programmed to draw 15 ul of borate buffer pH 10.4 then 3 µL of the sample and mix, then draw 2 µL from OPA reagent, mix with 10 µL of air, wait 2 minutes, then all 20 µL was injected. It is important to note if you store the OPA solution under Argon, the inventors found it was useable for 2 weeks and not just 3 days as suggested by the manufacture.

FIG. 1 provides an example of the amino acids the inventors were able to detect using the above method for analyzing amino acids they have published. Cunningham, G. et al., Development of a robust 30-minute reverse-phase high pressure liquid chromatography method to measure amino acids using widely available equipment and its comparison to current clinical ion-exchange chromatography measurement. MOLECULAR GENETICS AND METABOLISM REPORTS, 2022, 31, 100868, which is incorporated by reference for all purposes. Concentrations for the peaks are 100 uM each.

This method combines a simple sample preparation, using high pressure liquid chromatography instrument. This instrument itself can undergo multiply configurations, for various methods unlike the instruments used today that can only run amino acids. This method offers a much faster sample process time of 30 minutes, from the 2 or more hours in the traditional assay. it utilized 2 buffers instead of the traditional 5, so is more cost effective.

### Sample Preparation

Calculating intercellular volume and using this to calculate an unknown’s concentration: Given the real problem for analyzing a red blood cells sample is often they have clotted or frozen after being stored at -80 for months. One can measure the amount of amino acids in these samples but without a known intracellular volume you cannot calculate a concentration, like with cell lysate and tissue, most are forced to normalize by protein levels. Simple put how to solve for V₂ and C₂ when both are unknown. The inventors will present a method to calculate the amino acid concentration in these samples by calculating the intracellular volume first. The inventors cannot use C₁V₁ = C₂V₂ directly because they are working with two unknowns and one equation.

If each unknown could be addressed in multiple, but related equations, one could use the process of solving multiple unknowns using simultaneous equation to answer this problem. Remember how this works one needs multiple equations with each unknown represented in each equation.

$$\text{2X+3Y=25}$$

$$\text{4X+Y=15}$$

Use first equation to solve for X in terms of Y
If 2X+3Y=25
Then 2X=25-3Y
Therefore X= (25-3Y)/2

We can put this value for X in the second equation and solve for Y

$$\text{4} \left(\text{25-3Y}\right) / \text{2} \text{+Y=15}$$

$$\left(\text{100-12Y}\right) / \text{2} \text{+Y=15}$$

$$\text{50-15=5Y}$$

$$\text{Y=7}$$

Use this value for Y in either equation to solve for X and

$$\text{X=2}$$

One can set up an experiment of a known volume but would treat as unknowns to see how accurately one can calculate the volume and then its true concentration.

To a sample, add a known volume of the known concentration of your internal standard and see if one can calculate the sample’s volume. To a prepared “unknown” one would add 100 ul of 500 uM internal standard (IS), and run, integrate, and calculate.

$$\text{Using} {\text{C}}_{\text{1}} {\text{V}}_{\text{1}} {\text{=C}}_{\text{2}} {\text{V}}_{\text{2}}$$

C₁ is area or concentration of internal standard injection where V₁ and is the volume. Note the concentration and volume are a constant, 500 uM and 100 ul respectfully

$$\text{Then} \text{V2=} \left({\text{C}}_{\text{1}} {\text{V}}_{\text{1}}\right) / {\text{C}}_{\text{2}}$$

Remember V₂ or total is V₁ + Volume in sample and C1 and C2 are the concentration of the IS in the standard and the sample

$$\text{Vol} \text{in} \text{sample} \text{=} \left({\text{C}}_{\text{1}} {\text{V}}_{\text{1}} / {\text{C}}_{\text{2}}\right) \text{-} {\text{V}}_{\text{1}}$$

| Sample | V₁ | C₁ | V₂ | C₂ |
|---|---|---|---|---|
| | 100 | 500 | 100+X | 340 |

$${\text{V}}_{\text{2}} \text{=} \left(\left(\text{500}\right) \left(\text{100ul}\right) / \left(\text{340}\right)\right) \text{-100ul}$$

$${\text{V}}_{\text{2}} = \text{147ul-100ul} \text{or} \text{47ul} \text{for} \text{original} \text{sample} \text{volume} .$$

Measuring Protein amounts in RBC’s and the importance of using correct volume of solvent: blood is drawn fresh from a volunteer, centrifuge with the plasma removed in equal volume aliquots, then stored in -80 for later. Gently mix the remaining RBC’s and remove in equal volume aliquots and store in -80 for later. Done in triplicates, samples were diluted 1 to 2, 1 to 3, and 1 to 5 using PBS with 1 mM AABA, then homogenized.

Protein concentration was measured. The following results are in mg/ml.

| | Aliquot 1 | Aliquot 2 | Aliquot 3 | Avg mg protein/ml |
|---|---|---|---|---|
| 1 to 2 dilution | 28.99 | 26.19 | 27.59 | 27.59 |
| 1 to 3 dilution | 34.31 | 34.55 | 34.43 | 34.43 |
| 1 to 5 dilution | 20.55 | 20.59 | 22.48 | 21.20 |

So, if one multiplies the protein averages by the dilution 2, 3, and 5 one should get the same final concentration for each of the averages, because they were all from the same sample, but instead one does not.

| | Avg | DF | Final Conc mg/ml |
|---|---|---|---|
| 1 to 2 dilution | 27.59 | 2.00 | 55.18 |
| 1 to 3 dilution | 34.43 | 3.00 | 103.29 |

This is important because the reason you use protein concentration is to normalize the sample and if the protein concentration is incorrect, the final answer is also incorrect but one would not realize the error Why does this happen? The inventors consider that one must have sufficient solution when homogenizing samples, or the larger proteins do not have enough volume to go into solution. This would explain why the 1 to 3 and 1 to 5 dilutions give nearly the same the final concentration. Again, correcting for the dilution factor should give one the same amount of protein concentration for one is using enough buffer? The answer is one would not.

Calculating intercellular volume in RBC’s: In preparing fresh red blood cells, add an estimate of 2x (v/v) volume of PBS with protease inhibitors and a known concentration of our internal standard (IS) AABA, to the sample before it begins to thaw. Placed on ice for 5 minutes then vortex for 10 seconds. Centrifuge at 14000 rpm for 7 minutes. Supernatant was collected and transferred to a 3K spin filter column and centrifuged at 9,000 RPM for 20 minutes. This supernatant is then collected and ready for analysis.

We cannot use C₁V₁ = C₂V₂ because two unknowns and one equation, but if by adding a known volume of the known concentration of an internal standard (IS) one can calculate the sample’s volume based on it diluting the concentration of the (IS). C₁V₁=C₂V₂ where V₂= volume of IS added plus intracellular volume of the sample, C₁ is the concentration of the (IS) by itself and C₂ is the concentration of the (IS) in added to the sample.

$${\text{C}}_{\text{1}} {\text{V}}_{\text{1}} {\text{=C}}_{\text{2}} {\text{V}}_{\text{2}}$$

C₁ is the area for the peak for 1 mM AABA itself

V₁ is the amount added of this 1 mM AABA and add to the sample, here 100, 200 and 400 ul.

C₂ is the area of the peak for AABA in the sample diluted with the internal standard

V₂ is the volume in the sample (Vs) plus the V₁ addition.

1 to 2 dilution V₁ is 100 ul and V₂ is Vs₂ plus 100.

1 to 3 dilution V₁ is 200 ul and V₂ is Vs₃ plus 200.

1 to 5 dilution V₁ is 400 ul and V₂ is Vs₅ plus 400.

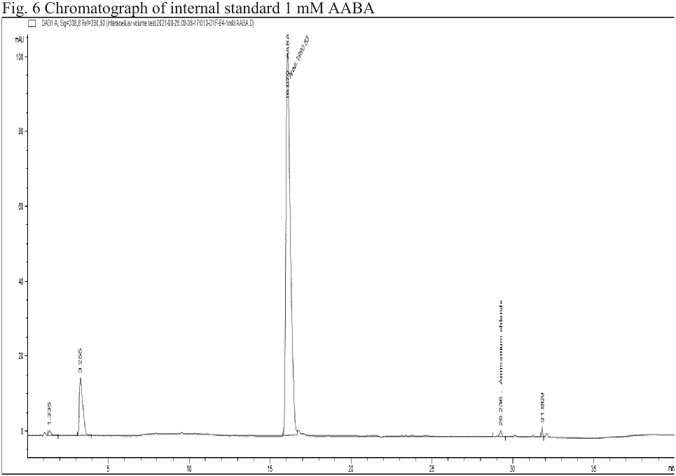

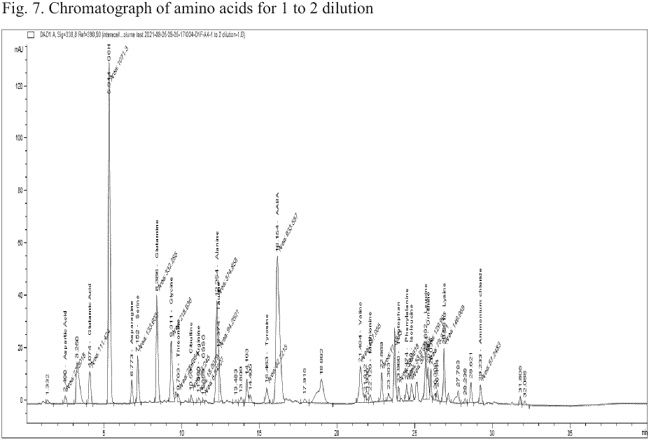

From FIGS. 6 and 7 one can calculate the IS concentration or area, C₁, in the standard injected along and used this solution to dilute and homogenize the sample. Then the inventors measured the IS concentration in the sample or area, C₂. FIG. 6 shows a chromatograph of internal standard 1 mM AABA and FIG. 7 shows a chromatograph of amino acids for 1 to 2 dilution.
C₁ is 1981.5
C₂ for the 1 to 2 dilution is 933
(1981.5)X(100 ul)=(933)X(Vsample2 + 100 ul)
Vsample2=113.3 ul
Calculate the dilution factor by (V₁+Vsample2)/Vsample2
(100+113.3)/113.3=1.9 not exactly 2 but very close

When one calculates the dilution factors for the other dilutions you get 2.6 and 4.7, not the expected 3 and 5, but again close. Now how to use this information to compare normalized to protein vs normalizing to AABA. The inventors used glutamine concentration from FIG. 3 as a test.

| | Protein mg/ml | Calculated Glutamine | Norm to protein | Norm to AABA |
|---|---|---|---|---|
| 1 to 2 | 55.2 | 334.0 uM | 12.1 umoles/mg | 634.6 uM |
| 1 to 3 | 103.3 | 248.1 uM | 7.2 umoles/mg | 645.06 uM |
| 1 to 5 | 106 | 141.6 uM | 6.7 umoles/mg | 665.52 uM |

A few more examples of some other amino acids from the same injection, whose concentration are calculated and normalized by the IS. The final concentrations for these amino acids at different dilutions are also in good agreement.

| Calculated Glycine | | Final Conc Norm to AABA | Calculated Cit | Final conc Norm to AABA |
|---|---|---|---|---|
| 1 to 2 | 218.6 uM | 411.6 uM | 23.2 uM | 43.7 uM |
| 1 to 3 | 157.8 uM | 407.6 uM | 16.9 uM | 43.7 uM |
| 1 to 5 | 91.8 uM | 430.6 uM | 8.9 uM | 42 uM |

Next, we apply this method to some frozen red blood cells to see if we can calculate the amino acid concentration under these conditions. We took whole blood and centrifuge for 7 minutes at 1000 rpms and collect the plasma in 200 ul aliquots, the buffy coat is removed, and the sample is mixed again before collecting the RBC’s in 200 ul aliquots. These were kept frozen at -80 for 1-2 months before analysis. We did two of these aliquots from each sample collected on the same day we added 400 ul of PBS with AABA. Although we collected the aliquots on the same day, we did the assay on two separate days to test and compare this method and results. As you can see the intracellular volume in a 200 ul sample of RBC, (V2-400) column averaged about 163 ul and 186 ul for the two subjects in table 1A.

Day 1

| | Area | V₂ | V2-400 | DF |
|---|---|---|---|---|
| IS | 5162.53 | | | |
| Y CTL | 3677.2 | 561.57 | 161.57 | 3.48 |
| K CTL | 3513.4 | 587.75 | 187.75 | 3.13 |

Day 2

| | Area | V2 | V2-400 | DF |
|---|---|---|---|---|
| 1 mM | 3614.51 | | | |
| Y CTL | 2563.5 | 564 | 164 | 3.44 |
| K CTL | 2477 | 583.7 | 183.69 | 3.18 |

Table 1A intracellular volumes in RBC’s

Furthermore, if you multiply this dilution factor by the concentrations of amino acids as we did for some selected amino acids you get a consistent intercellular concentration validating this method.

TABLE 1B

| | Test 1 | | | Test 2 | |
|---|---|---|---|---|---|
| | Concentration in uM | | | Concentration in uM | |
| | Y | K | | Y | K |
| Glutamic Acid | 36.9 | 68.09 | Glutamic Acid | 41.31 | 78.09 |
| Glutamine | 87.4 | 137.22 | Glutamine | 87.02 | 139.4 |
| Valine | 46.17 | 39.99 | Valine | 41.41 | 36.37 |
| Ornithine | 66.54 | 49.81 | Ornithine | 81.75 | 61.24 |

Sample preparation or Cell Lysate. To determine the levels of primary amino acids in cell lysate, HepG2 cells were cultured until confluent. Cells are then passaged and counted with 6 million cells per 6 mls of media transferred to 100 mm disk and left at 37 degrees incubator overnight. The next day the media was removed and washed twice with 10 mls of PBS. We then by using a 200 ul pipet would tilt the plate and pipet out as much as the remaining liquid as possible. The plates are sealed with parafilm and placed in a -80 overnight. The inventors have found this lyses all the cells and it is easy to collect the intracellular fluid. The next day, or longer, the plate can remain in the -80 for days, they are removed and placed on the bench at a slight angle to thaw. Once the cells have thawed, add 200 ul of 1 mM AABA in PBS and using a cell scraper, scrap the cell and collect as much volume as possible before centrifuging this liquid at 14000 rpm for 10 minutes. Supernatant was collected then transferred to a 3K spin column filter and centrifuged at 8,500 RPM for 25 minutes. The filtrate is then collected and prepped for amino acid analysis. FIG. 4 is an example of the chromatograph of one of these HepG2 samples.

Tables 3A and 3B show the integrations of the internal standard and the final calculation of the intercellular volumes and the dilution factor for each cell lysate. Tables of the protein concentrations for each sample and the calculations for some of the amino acids based on each injection are also provided in these tables where GSH = Glutathione.

TABLE 3A

| Area of AABA in standard | 1508.25 | | | | |
|---|---|---|---|---|---|
| | Sample 1 | Sample 2 | Sample 3 | Sample 4 | Sample 5 |
| Area of ABBA in samples | 861.30 | 900.51 | 943.31 | 877.21 | 854.65 |
| Vtotal calculation | 350.23 | 334.98 | 319.78 | 343.87 | 352.95 |
| V2 calculation | 150.23 | 134.98 | 119.78 | 143.87 | 152.95 |
| DF | 2.33 | 2.48 | 2.67 | 2.39 | 2.31 |
| | Sample 1 | Sample 2 | Sample 3 | Sample 4 | Sample 5 |
| Protein amounts for each sample mg/ml | 10.87 | 10.01 | 9.94 | 10.84 | 9.48 |
| | Raw Concentrations for each sample based on AA STD uM | | | | |
| | Sample 1 | Sample 2 | Sample 3 | Sample 4 | Sample 5 |
| Aspartic Acid | 98.88 | 99.88 | 96.10 | 95.54 | 89.18 |
| Glutamic Acid | 197.39 | 213.44 | 204.71 | 209.37 | 204.15 |
| GSH | 303.11 | 281.21 | 284.10 | 287.87 | 273.03 |
| Lysine | 12.00 | 12.84 | 12.30 | 11.27 | 10.36 |

TABLE 3B

| | Final concentrations based on normalizing to Internal StduM | | | | |
|---|---|---|---|---|---|
| | Sample 1 | Sample 2 | Sample 3 | Sample 4 | Sample 5 |
| Aspartic Acid | 230.531 | 247.881 | 256.551 | 228.351 | 205.801 |
| Glutamic Acid | 460.18 | 529.71 | 546.53 | 500.42 | 471.09 |
| GSH | 706.64 | 697.89 | 758.48 | 688.05 | 630.05 |
| Lysine | 27.97 | 31.87 | 32.83 | 26.94 | 23.90 |

| | Final concentrations based on normalizingto protein uM/mg | | | | |
|---|---|---|---|---|---|
| | Sample 1 | Sample 2 | Sample 3 | Sample 4 | Sample 5 |
| Aspartic Acid | 9.09 | 9.98 | 9.67 | 8.81 | 9.40 |
| Glutamic Acid | 18.15 | 21.33 | 20.59 | 19.31 | 21.53 |
| GSH | 27.87 | 28.10 | 28.58 | 26.55 | 28.79 |
| Lysine | 1.10 | 1.28 | 1.24 | 1.04 | 1.09 |

As shown above, the inventors have presented a method to quantify the intercellular volumes of the most common amino acids based on our previously published assay; see Cunningham, et al. Development of a robust 30-minute reverse-phase high pressure liquid MOLECULAR GENETICS AND METABOLISM REPORTS chromatography method to measure amino acids using widely available equipment and its comparison to current clinical ion-exchange chromatography measurement. 31(2022) 10086.

The inventors showed that this method works for calculating the intercellular volume and then the concentration of amino acids in RBC sample even after it has been frozen or clotted. It is also useful for calculating the amino acid concentration in cell lysates. Remember the normal way of calculating amino acid involves normalizing to protein which gives units in umoles/mg of protein, being able to calculate the intercellular volume, it will give one units in uM which is the same units used for plasma amino acids, making this is the first method that allows one to make a direct comparison of plasma and red blood cells in a sample.

Detailed Catalog of Materials Used

| | |
|---|---|
| Part Number | Company |
| AA standard | |
| Amino Acid Standard | AAS18-10ML Sigma |
| L-Amino Acids | LAA21-KT Sigma |
| L-Glutathione oxidized | G4376-5G Sigma |
| L-Glutathione reduced | G6529-5G Sigma |
| L-Allo-Isoleucine | I8754-100MG Sigma |
| Beta Alanine | PHR1349-1G Sigma |
| γ-Aminobutyric acid | 03835-250MGSigma |
| L-Ornithine monohydrochloride | O6503-25G Sigma |
| L-Homocystine | H6010-100MG Sigma |
| L-Citrulline | C7629-100G Sigma |
| Taurine | 166541000 ACROS |
| D-2-Aminobutyric acid (AABA) | 116122-5G Sigma |
| Derivatization Reagent | |
| O-Phthaldialdehyde Reagent Solution (OPA) | 5061-3335 Agilent |
| Borate Buffer | 5061-3339 Agilent |
| Column | |
| Poroshell HPH-C18, 3.0×100 mm 2.7 um | 695975-502 Agilent |
| UHPLC Grd, Poroshell HPH-C18, 3.0 mm | 823750-928 Agilent |
| Buffer 1 | |
| Sodium phosphate dibasic | S9763-1KG Sigma |
| Sodium azide | S8032-100G Sigma |
| Sodium tetraborate decahydrate | S9640-500G Sigma |
| Buffer 2 | |
| Acetonitrile HPLC Grade BDH83639.400 VWR | |
| Methanol HPLC Grade | BDH20864.400 VWR |
| Water HPLC Grade | BDH23595.400 VWR |
| Misc. | |
| Hydrochloric acid 5.0N | BDH7419-1 VWR |
| Phosphoric Acid | BDH3104-2.5PLC VWR |
| VWR Spin filter 3k | 82031-346 VWR |
| Cap screw blue | 97052-794 VWR |
| Insert MS plastic spring | 97051-410 VWR |
| Vials (Glass) | 5182-0714 Agilent |
| Vials (Amber borosilicate) | 5182-0716 Agilent |

All publications and patent applications mentioned in this specification are herein incorporated by reference in their entirety to the same extent as if each individual publication or patent application was specifically and individually indicated to be incorporated by reference, especially referenced is disclosure appearing in the same sentence, paragraph, page or section of the specification in which the incorporation by reference appears.

The citation of references herein does not constitute an admission that those references are prior art or have any relevance to the patentability of the technology disclosed herein. Any discussion of the content of references cited is intended merely to provide a general summary of assertions made by the authors of the references and does not constitute an admission as to the accuracy of the content of such references.

1. A method for detecting amino acids in a sample volume of 50 µL or less comprising:
(a) removing proteins from a sample,
(b) derivatizing the sample obtained from (a) by contacting it with O-Phthalaldehyde (OPA),
(c) injecting the derivatized sample from (b) into a RP-HPLC column to produce fractions, and
(d) detecting RP-HPLC resolved fractions or components as they elute from the RP-HPLC column by ultraviolet illumination.

2. The method of claim 1, wherein the sample has a volume of 25 µL or less.

3. The method of claim 1, wherein the sample is obtained from a subject with an in-born error of metabolism including maple syrup urine disease, phenylketonuria, organic acidemias, homocystinuria, tyrosinemia, and urea cycle disorders.

4. The method of claim 1, wherein the sample comprises blood, plasma, or serum.

5. The method of claim 1, wherein the sample comprises cerebral spinal fluid, synovial fluid, lymph, peritoneal fluid, amniotic fluid, saliva, breast milk, gastric juice, bile, perspiration, tears, semen, vaginal secretions, breast milk, ascitic fluid, mucous, urine or pus.

6. The method of claim 1, wherein (a) the removing of proteins from the sample comprises spin filtering the sample without dilution or acid precipitation.

7. The method of claim 1, wherein (b) derivatizing the sample comprises derivatizing the sample from (a) with O-Phthalaldehyde (OPA) on an RT-HPLC injector needle immediately before injection into the column.

8. The method of claim 1, wherein in (c) the mobile phase is a binary mobile phase (A) and (B); wherein (A) comprises a mixture of sodium phosphate, sodium borate, and sodium azide, and (B) comprises a mixture of acetonitrile, methanol and water.

9. The method of claim 1, wherein (c) comprises RP-HPLC chromatography comprising a run time of 30 minutes or less.

10. The method of claim 1, wherein (d), detecting the fractions comprises illuminating the fractions with UV light at a wavelength of 338 nm + 20 nm.

11. A method for simultaneous determination of ammonium and primary amino acids in a same sample comprising:
(a) removing solids from a sample,
(b) derivatizing the sample obtained from (a) by contacting it with O-Phthalaldehyde (OPA),
(c) injecting the derivatized sample from (b) into a RP-HPLC column to produce fractions, and
(d) detecting RP-HPLC resolved fractions or components as they elute from the RP-HPLC column by ultraviolet illumination.

12. The method of claim 11, wherein the sample has a volume of 25 µL or less.

13. The method of claim 11, wherein the sample is obtained from a subject with an in-born error of metabolism including maple syrup urine disease, phenylketonuria, organic acidemias, homocystinuria, tyrosinemia, and urea cycle disorders.

14. The method of claim 11, wherein the sample comprises plasma.

15. The method of claim 11, wherein the sample comprises cerebral spinal fluid, synovial fluid, lymph, peritoneal fluid, amniotic fluid, saliva, breast milk, gastric juice, bile, perspiration, tears, semen, vaginal secretions, breast milk, ascitic fluid, mucous, urine or pus.

16. The method of claim 11, wherein (a) the removing of proteins from the sample comprises spin filtering the sample without dilution or acid precipitation.

17. The method of claim 11, wherein (b) derivatizing the sample comprises derivatizing the sample from (a) with O-Phthalaldehyde (OPA) on an RT-HPLC injector needle immediately before injection into the column.

18. The method of claim 1, wherein (c) comprises RP-HPLC chromatography comprising a run time of 30 minutes or less.

19. The method of claim 1, wherein (d), detecting the fractions comprises illuminating the fractions with UV light at a wavelength of 200-400 nm.

20. The method of claim 1, wherein (d), detecting the fractions comprises illuminating the fractions with UV light at a wavelength of 338 nm ± 20 nm.

21. A method for calculating an intercellular volume of cells used to produce a cell lysate, comprising:
(a) producing a cell lysate,
(b) adding a known concentration of a non-naturally occurring amino acid to the cell lysate to produce a sample for RP-HPLC,
(c) derivatizing the sample obtained from (b) by contacting it with O-Phthalaldehyde (OPA),
(d) injecting the derivatized sample from (c) into a RP-HPLC column to produce fractions,
(e) detecting RP-HPLC resolved fractions or components as they elute from the RP-HPLC column by ultraviolet illumination; and
(f) calculating an average intracellular volume of the cells used to produce the lysate by comparison of the concentration of the non-naturally occurring amino acid and the detected concentrations of one or more analytes with the concentration of the non-naturally occurring amino acid, thereby calculate an average cell volume of the cells used to produce the cell lysate.

22. The method of claim 21, wherein the sample has a volume of 50 µL or less.

23. The method of claim 21, wherein the cells comprise red blood cells (RBCs).

24. The method of claim 21, wherein the cells comprising leukocytes or comprises cells from a somatic tissue.

25. The method of claim 21, further comprising removing proteins from the cell lysate prior to (b) or (c).

26. The method of claim 21, further comprising spin filtering the cell lysate without dilution or acid precipitation prior to (b) or (c).

27. The method of claim 21, wherein (b) derivatizing the sample comprises derivatizing the sample from (a) with O-Phthalaldehyde (OPA) on an RT-HPLC injector needle immediately before injection into the column.

28. The method of claim 21, wherein in (d) the mobile phase is a binary mobile phase (A) and (B); wherein (A) comprises a mixture of sodium phosphate, sodium borate, and sodium azide, and (B) comprises a mixture of acetonitrile, methanol and water.

29. The method of claim 21, wherein (d) comprises RP-HPLC chromatography comprising a run time of 30 minutes or less.

30. The method of claim 21, wherein (e), detecting the fractions comprises illuminating the fractions with UV light at a wavelength of 338 nm ± 20 nm. 200-400.
